(19) United States
(12) Patent Application Publication
(10) Pub. No.: US 2023/0118632 A1
(43) Pub. Date: Apr. 20, 2023

(54) OPHTHALMIC CANNULA

(71) Applicant: Nova Eye, Inc., Fremont, CA (US)

(72) Inventors: Mark Arnoldussen, Fremont, CA (US); Steve Burnett, Fremont, CA (US); Jordan Green, Kent Town (AU); Victor Previn, Kent Town (AU)

(21) Appl. No.: 17/506,407

(22) Filed: Oct. 20, 2021

(51) Int. Cl. A61F 9/007; A61B 3/00

(52) U.S. Cl. CPC A61F 9/00781; A61B 3/0008

(57) ABSTRACT

A microsurgical instrument includes a cannula with a straight segment at a proximal end and a parting tip at a distal end. The parting tip includes a parting tip projection, a convex parting edge formed on the parting tip projection, and a spatulated parting face. The spatulated parting face includes a convex surface portion formed on the parting tip projection and a concave surface portion joined to the convex surface portion along a line of inflection at a proximal end of the parting tip projection. The microsurgical instrument optionally includes a cannula head attached to the cannula. The cannula head includes a tapered outer surface, a circumferential outer flange, an arcuate inner flange, and an inner flange ridge extending radially away from the inner flange. A payload guide attached to the cannula and cannula head directs payloads into the lumen of the cannula.

### FIELD OF THE INVENTION

Embodiments are related to surgical instruments having a hollow tubular element configured for insertion into a human eye.

### BACKGROUND

The flow of aqueous humor passing through the trabecular meshwork, Schlemm's canal, collector channels, and other parts of the drainage network of an eye may be impeded by an obstruction or collapse of part of the drainage network, possibly increasing intraocular pressure sufficiently to degrade vision. Surgical treatments may be used to improve the flow of aqueous humor and reduce intraocular pressure. Some surgical treatments form an opening in the sclera or cornea and insert a small trocar or cannula through the opening into Schlemm's canal or another part of the drainage network. After the cannula is inserted into the eye, a payload may be passed through the lumen of the cannula to remove obstructions, force apart the walls of a collapsed drainage channel, or for other reasons. Some examples of a payload include a viscoelastic fluid, a flexible microcannula, a fiber optic, a stent, forceps, a scalpel, a polymer fiber, and a metallic wire. It may be preferable to deliver a payload including a solid object into Schlemm's canal tangentially to the interior wall surfaces of the canal to prevent the payload from damaging tissues forming the canal wall and/or prevent the payload entering directly into a collector channel instead of following the cavity wall.

Some cannulas are provided with a cutting tip at the distal end of a straight, hollow, tubular element. When passing through the lumen of a cannula with a straight tubular element, a payload such as a flexible microcannula may not bend to conform to the curved walls of Schlemm's canal until the microcannula exits the lumen of the cannula and impacts the tissue forming the canal wall. The force required to bend the payload may therefore applied to the tissue. Small deviations in the cannula entry angle relative to the walls of Schlemm's canal may cause substantial increases in the bending forces applied to the tissue. A cannula with a straight tubular element near the distal end may be used with an associated marking tool that places a mark on the sclera to indicated a preferred point of entry and/or angle of entry of the cannula into Schlemm's canal to improve the likelihood of achieving tangential entry of a payload into Schlemm's canal.

Some cannulas place a cutting tip at the end of a curved or bent hollow tubular element. The curved tubular element may induce bending in a payload such as a microcannula before the payload exits the cannula, possibly improving tangential entry of the payload into Schlemm's canal. Schlemm's canal has an elongate cross-sectional shape and an approximately circular outer perimeter near the posterior part of the corneoscleral junction. The circular outer perimeter may be described by a radius from the center of the iris to the outer wall of Schlemm's canal. However, a tubular element that has a bend radius substantially different from the radius of Schlemm's canal may cause non-tangential entry of a payload into Schlemm's canal. Some cannulas with a curved tubular element are configured to deliver stents, ocular implants, or other devices that are too large to fit into Schlemm's canal. Using such cannulas to enter Schlemm's canal may require larger openings through eye tissues compared to an instrument designed for use in Schlemm's canal.

### SUMMARY

An example apparatus embodiment includes a cannula formed as a hollow tube. The cannula includes a straight segment at a proximal end of the cannula and a parting tip at a distal end of the cannula. An example parting tip includes a parting tip projection; a convex parting edge formed on the parting tip projection; and a spatulated parting face. An example spatulated parting face includes a convex surface portion formed on the parting tip projection; and a concave surface portion joined to the convex surface portion along a line of inflection at a proximal end of the parting tip projection.

The example cannula embodiment optionally further includes the convex surface portion of the spatulated parting face extending from the convex parting edge to the line of inflection. The example cannula embodiment optionally includes the concave surface portion extending to an external surface of the cannula.

Another example apparatus embodiment includes a cannula head attached to the cannula. An example cannula head includes an exterior wall having a tapered outer surface; a circumferential outer flange attached to the outer surface at a proximal end of the cannula head; an arcuate inner flange extending proximally away from the exterior wall, the inner flange having an outer surface; an inner flange ridge extending radially away from the outer surface of the inner flange; and a payload guide support wall joined to an interior surface of the exterior wall. The example apparatus embodiment further includes a payload guide attached to the spatulated cannula, with the payload guide slidably engaged with the payload guide support wall.

### BRIEF DESCRIPTION OF THE DRAWINGS

FIG. 1 is a pictorial view toward a distal end of an example apparatus embodiment of a microsurgical instrument including a spatulated cannula and a cannula head.

FIG. 2 is a pictorial view toward a proximal end of the example microsurgical instrument of FIG. 1.

FIG. 3 is a side view of the example microsurgical instrument of FIGS. 1-2, illustrating an example of a cannula head with an optional cannula orientation ridge and a cannula with an optional arcuate segment interposed between a parting tip and a straight segment.

FIG. 4 is a view toward a spatulated parting face on the parting tip at the distal end of the spatulated cannula of the example microsurgical instrument of the previous figures.

FIG. 5 shows a side view of the example microsurgical instrument having a cannula with the parting tip attached directly to the straight segment without an intervening arcuate segment.

FIG. 6 shows a view downward onto the top side of the example microsurgical instrument of FIG. 5.

FIG. 7 is a cross-sectional view A-A of an example cannula head. A location and viewing direction for the cross-sectional view is marked by section arrows A-A in FIG. 6.

FIG. 8 shows a view toward the proximal end of the example microsurgical instrument, illustrating examples of the outer flange and inner flange of the cannula head and a payload guide attached to the cannula.

FIG. 9 shows a view toward the distal end of the example microsurgical instrument of FIGS. 1-4, illustrating an example of a cannula housing with an optional cannula orientation ridge and a cannula with an arcuate segment near the distal end.

FIG. 10 shows a view toward the distal end of the example microsurgical instrument having the parting tip attached directly to the straight segment of the cannula as in the examples of FIGS. 5-6, and further illustrating an example of a cannula head formed without the optional cannula orientation ridge.

FIG. 11 shows a side view of an example spatulated cannula having an optional arcuate segment interposed between the parting tip and the straight segment.

FIG. 12 shows a side view of an example spatulated cannula having the parting tip attached directly to the straight segment without an intervening arcuate segment.

FIG. 13 shows a view toward the spatulated parting face 150 and the bottom side 242 of the example spatulated cannulas, representing cannulas with the parting tip 112 attached to an arcuate segment and cannulas with the parting tip attached to the straight segment.

FIG. 14 shows a cross-sectional view B-B of the spatulated cannula examples. Locations and viewing directions for section B-B are marked by section lines B-B in FIG. 13.

FIG. 15 shows a partial pictorial view an example parting tip, illustrating examples of the convex surface portion and the concave surface portion of the spatulated parting face in accord with parting tips on all cannula embodiments disclosed herein.

FIG. 16 shows a cross-sectional view C-C of an example parting tip applicable to all cannula embodiments disclosed herein. An example location and viewing direction for section C-C is marked in FIG. 12.

FIG. 17 shows an alternative cross-sectional view C-C illustrating examples of a light source 224 protruding from the lumen of an example spatulated cannula, without the light source interfering with the effectiveness of the parting tip projection for penetrating tissue such as the sclera of an eye.

FIG. 18 shows a cross-sectional view through the lumen of a cannula having a bevel face at the distal end and a light source protruding outside the lumen beyond a cutting edge of the bevel face, further illustrating an example of the light source interfering with the effectiveness of the cutting edge of the bevel face for penetrating tissue. (PRIOR ART)

FIG. 19 continues the example of FIG. 18. showing the example light source retracted into the interior of the lumen so as to avoid interfering with the effectiveness of the cutting edge of the bevel face for penetrating tissue, resulting in reduced visibility of the distal end of the light source and the distal end of the cannula. (PRIOR ART)

### DESCRIPTION

Example apparatus embodiments of a microsurgical instrument include a spatulated cannula formed from rigid hollow tubing, with a parting tip at the distal end of the cannula, an optional arcuate segment having a radius of curvature approximately equal to the radius of curvature of Schlemm's canal in a human eye, and a straight segment extending from the arcuate segment to the proximal lend of the cannula. Some embodiments of the spatulated cannula do not include the optional arcuate segment, instead having the parting tip attached directly to the straight segment. Some apparatus embodiments further include a cannula head strongly attached to the cannula. The cannula head preferably includes mounting features enabling rotatable attachment of the cannula head to a handpiece to enable a surgeon to select and maintain a preferred orientation of the parting tip relative to the handpiece. The spatulated cannula includes features which enhance the visibility of the parting tip as it penetrates eye tissue and enters parts of the eye's drainage system such as Schlemm's canal, without interfering with the effectiveness of the parting tip for penetrating the eye tissue.

Microsurgical Instrument embodiments are effective for delivering solid and/or liquid payloads into vessels and chambers in an eye with minimal disruption of eye tissues. The parting tip of the cannula includes a spatulated parting face having a concave portion and a convex portion smoothly joined along a line of inflection to the concave portion. The convex portion, formed on a parting tip projection extending out from the concave portion, extends to a convex parting edge at the extreme distal end of the spatulated cannula. The convex parting edge on the parting tip projection readily penetrates the sclera and other tissues. The convex and concave portions of the spatulated parting face gently stretch and force open tissue penetrated by the convex parting edge, or alternate instrument, until the spatulated cannula is able to enter Schlemm's canal or another chosen structure. In contrast to previously known cannulas with trocar tips, circular razor edges, lancet points, bevel faces, and other tip designs that for the most part cut rather than stretch tissue, the disclosed spatulated parting tip embodiments cause less bleeding, promote faster healing, and reduce a risk of infection.

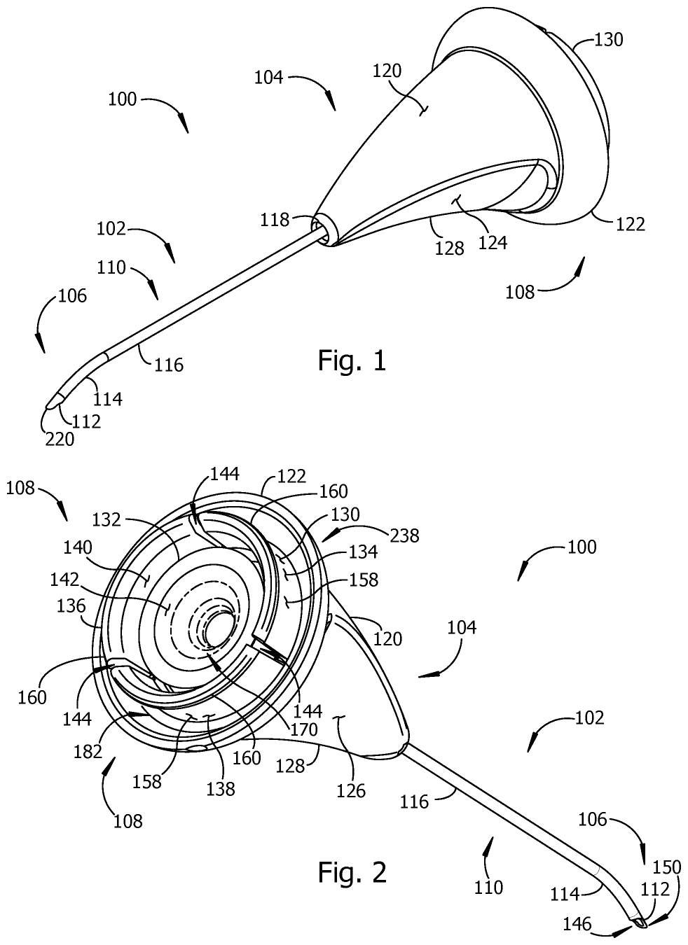

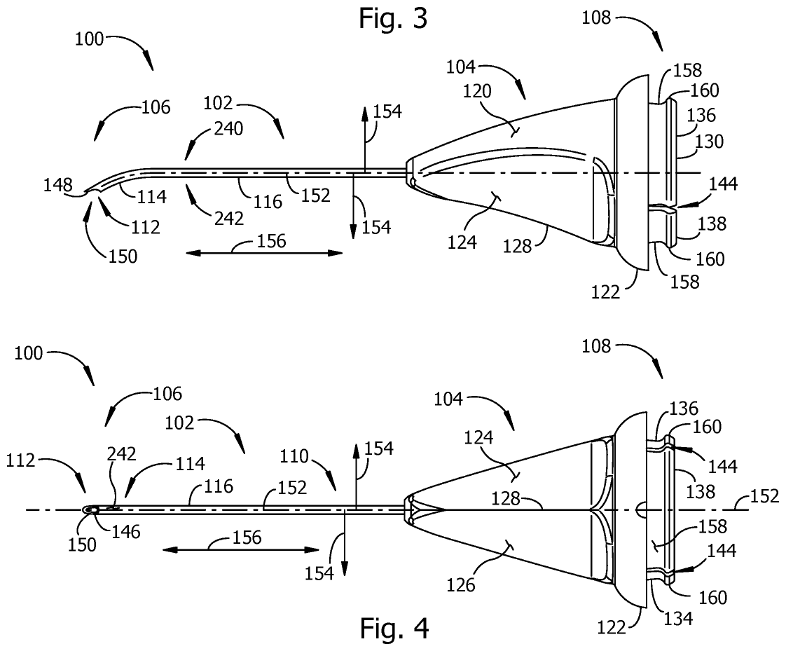

FIG. 1 and FIG. 2 are pictorial views of an example embodiment of the microsurgical instrument 100 including a spatulated cannula 102 attached to a cannula head 104. FIG. 3 and FIG. 4 are orthographic projections of the example microsurgical instrument 100 of FIG. 1 and FIG. 2. All disclosed embodiments of the spatulated cannula 102 are formed from a hollow tubular member 110 having a spatulated parting face 150 formed on a parting tip 112 at a distal end 106 and a lumen 146 extending through the spatulated parting face to a proximal end 108 of the tubular member. The hollow tubular member 110 is preferably made from a rigid material such as a surgical grade of stainless steel having a flexural rigidity of at least 1.5×10⁻⁸ kN-m², stiff enough to readily penetrate the sclera and other tissue in an eye without the hollow tubular member 110 flexing sufficiently to interfere with accurate placement and/or rotational orientation of the spatulated cannula in eye tissue.

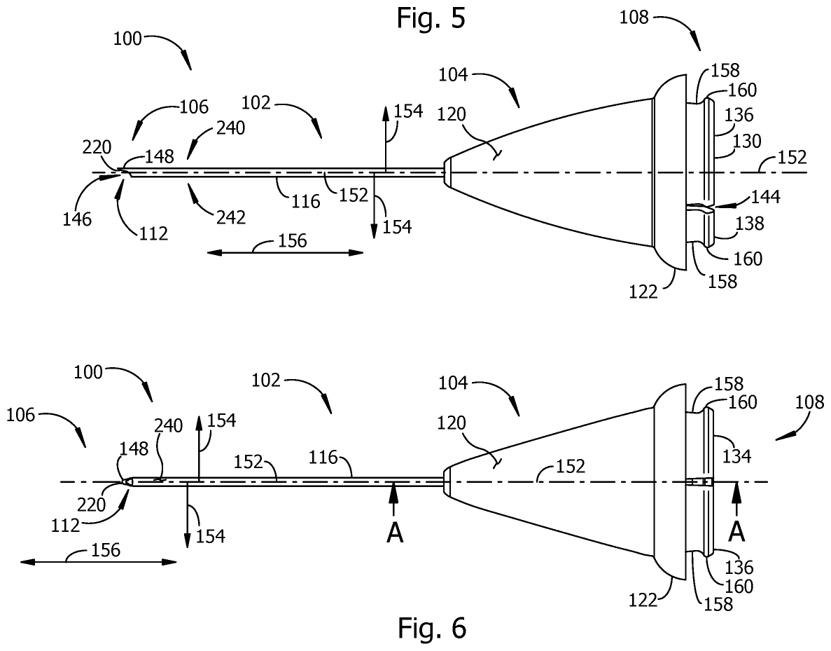

The example spatulated cannula 102 in FIGS. 1-4 includes a parting tip 112 attached to, or alternatively formed as an integral part of, an optional arcuate segment 114 of the tubular member 110. The arcuate segment 114 is attached to or alternatively formed as an integral part of an elongate straight segment 116 of the tubular member 110. A spatulated cannula 102 including the optional arcuate segment 114 may be referred to as a curved spatulated cannula 102. As will be described for the examples of a spatulated cannula in FIGS. 5-6, the optional arcuate segment may not be included and the parting tip 112 may instead be joined directly to the straight segment 116. A spatulated cannula 102 having the parting tip joined directly to the straight segment may be referred to as a straight spatulated cannula 102.

Some apparatus embodiments 100 include a cannula head 104 attached to a curved spatulated cannula 102 as in the examples of FIGS. 1-4, or alternatively to a straight spatulated cannula 102 as in the examples of FIGS. 5-6. As suggested in the examples of FIGS. 1-10, the cannula head 104 optionally included with some embodiments 100 is formed with a tapered outer surface 120 joined to, or alternatively formed as an integral part of, a circumferential outer flange 122. A channel 182 is formed between the circumferential outer flange 122 and an outer surface 158 of an inner flange 130. The outer flange 122, channel 182, and inner flange 130 are parts of a handpiece connector 238 for firmly connecting the cannula head to a handpiece (not illustrated).

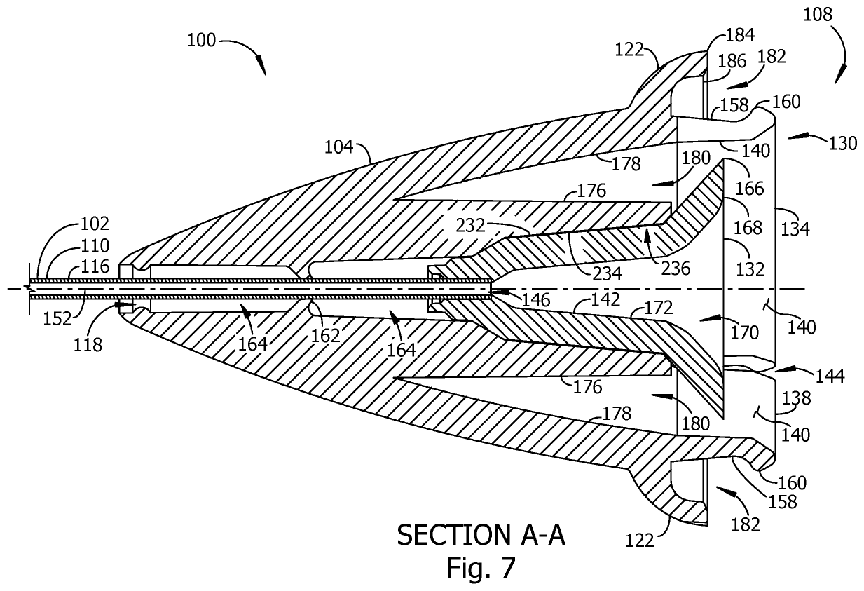

For microsurgical instrument embodiments 100 including a cannula head 104 as in the examples of FIGS. 1-10, the straight segment 116 of the spatulated cannula 102 passes through an aperture 118 at the distal end of a tapered outer surface 120. The spatulated cannula 102 is retained in the cannula head 104 by a payload guide 132 strongly attached to the tubular member 110 by adhesive. The payload guide 132 rests firmly against a support wall in the cannula head, as will be explained in more detail with regard to FIG. 7. An inner surface 142 of the payload guide 132 forms a boundary of a void space 170 in the payload guide. The void space 170 in the payload guide 132 is in fluid communication with the lumen 146 of the spatulated cannula 102. The tapered inner surface 142 encourages a payload entering the open proximal side of the payload guide to enter the proximal end of the lumen 146. An example payload such as a flexible microcannula (not illustrated) Inserted into the open proximal side of the payload guide 132 will be encouraged by sliding contact with the tapered surface 142 to move toward and enter the lumen 146 of the spatulated cannula 102.

The Inner flange 130 optionally includes an inner flange ridge 160 extending radially outward from an outer surface 158 of the inner flange. The inner flange ridge 160 is positioned to engage a corresponding circumferential channel on a handpiece (not illustrated) configured to receive the cannula head 104. The inner flange may optionally be formed with at least two flange segments separated from one another by flange slots. The flange segments are capable of flexing radially independently of one another, thereby providing for the inner flange to flex slightly when the inner flange ridge 160 engages a handpiece with a snap fit. The flange segments may also flex when the snap fit is interrupted to separate the cannula head from the handpiece. For the cannula head 104 examples of the figures, the inner flange 130 includes a first inner flange segment 134, a second inner flange segment 136 separated from the first inner flange segment 132 by an intervening first inner flange slot 144, and a third inner flange segment 138. The third inner flange segment 138 is separated from the second inner flange segment 136 by a second inner flange slot 144. A third inner flange slot 144 separates the first inner flange segment 134 and the third inner flange segment 138.

A cannula head 104 may optionally be formed with a first inset face 124 and a second inset face 126 intersecting one another along a cannula orientation ridge 128. As suggested in the examples of FIG. 3 and FIG. 4, a longitudinal centerline 152 is radially centered in the lumen 146 through the parting tip 112, arcuate segment 114, and straight segment 116 of the curved spatulated cannula 102. The cannula orientation ridge 128 is preferably coplanar with the longitudinal centerline 152 of the curved spatulated cannula. The bend direction of the arcuate segment 114, i.e. the concave side of the bend, is preferably toward the cannula orientation ridge 128. The optional first inset face 124, second inset face 126, and cannula orientation ridge 128 therefore give a clear visual and/or tactile indication to a person using an embodiment 100 of the plane and direction of bend of the arcuate segment 114 and the parting tip 112, which may otherwise be difficult to determine after the cannula 102 has been inserted through obscuring eye tissue such as the sclera.

An example cannula head 104 formed without the optional inset faces (124, 126) and cannula orientation ridge 128 is shown in a side view in FIG. 5 and a view toward the top side 240 in FIG. 6. FIGS. 5-6 also show examples of a straight spatulated cannula 102, with the spatulated parting tip 112 attached directly to the straight segment 116 of the tubular member 110.

FIGS. 3-6 further illustrate examples of reference directions used herein. A radial direction 154 is a direction perpendicular to the longitudinal centerline 152. A longitudinal direction 156 is a direction parallel to the portion of the longitudinal centerline through the straight segment 116.

Cross-section A-A in FIG. 7 shows examples of additional features of an embodiment of a microsurgical instrument 100 including the spatulated cannula 102 and cannula head 104. The longitudinal centerline 152 passes through the straight segment 116 of the spatulated cannula 102. The spatulated cannula 102 enters the cannula head 104 through an aperture 118 and extends into the payload guide 132, where the lumen 132 is in fluid communication with the void space 170 bounded by the sloped interior surface 142 of the tapered wall 172.

The tapered wall 172 of the payload guide 132 extends to a proximal surface on the payload guide having an outer circumferential edge 166 and an inner circumferential edge 168. In the example of FIG. 7, the outer circumferential edge 166 is spatially separated from the inner surfaces 140 of the inner flange 130. In an alternative arrangement, the outer circumferential edge and the inner surfaces 140 contact one another. The void space 170 is preferably open to the proximal side of the cannula head 104 to allow payloads to enter the payload guide.

When the payload guide 132 is installed in the cannula head 104, an outer surface 234 of the payload guide 132 contacts an inner surface 232 of a payload guide support wall 176. The outer surface 234 of the payload guide and the inner surface 232 of the support wall may each be formed with a Morse taper 236 to establish a secure fit between the payload guide 132 and the support wall 176. The cannula head 104 may optionally be formed with a void space 180 between the payload guide support wall 176 and an exterior wall 178 of the cannula head 104. The void space 180 may optionally be left open to the proximal side of the cannula head 104, or may alternately be filled with epoxy or molded as a solid section integral with the exterior wall 178 and the payload guide support wall 176.

As suggested in the example of FIG. 7, the outer flange 122 is formed with an outer circumferential edge 184 and an inner circumferential edge 186. A gap or circumferential channel 182 between the inner circumferential edge 186 and the inner flange ridge 160 on the inner flange 130 is positioned to receive an outer projection of a connector flange (not illustrated) extending from the distal end of a handpiece configured to accept the cannula head 104. The inner flange ridge 160 on the inner flange 130 is positioned to snap around an inner projection of the connector flange of the handpiece, the outer flange and inner flange on the cannula head establishing a rotatable yet firm press fit connection to the handpiece.

The press fit connection may be characterized by the torque needed to rotate the cannula head relative to the handpiece. Preferred values of rotational torque for rotating the cannula head relative to the handpiece have been measured to fall in a range from 0.01 Newton-meter to 0.03 Newton-meter. Values below 0.01 Newton-meter may allow unintended rotation of the cannula head. Values above 0.03 Newton-meter may increase the difficulty of precisely adjusting the rotational position of the cannula head and/or the cannula angle. For some example embodiments 100, a value of rotational torque for rotating the cannula head relative to the handpiece is 0.015 Newton-meter.

FIG. 7 further illustrates an example of one or more void spaces 164 formed in the cannula head 104. The straight segment 116 of the spatulated cannula 102 passes into the cannula head 104 through the aperture 118 and the void spaces 164. A circumferential cannula support flange 162 supports the spatulated cannula 102 in the void spaces 164. The void spaces 164 may optionally be filled with epoxy to oppose separation of the spatulated cannula 102 from the cannula head 104.

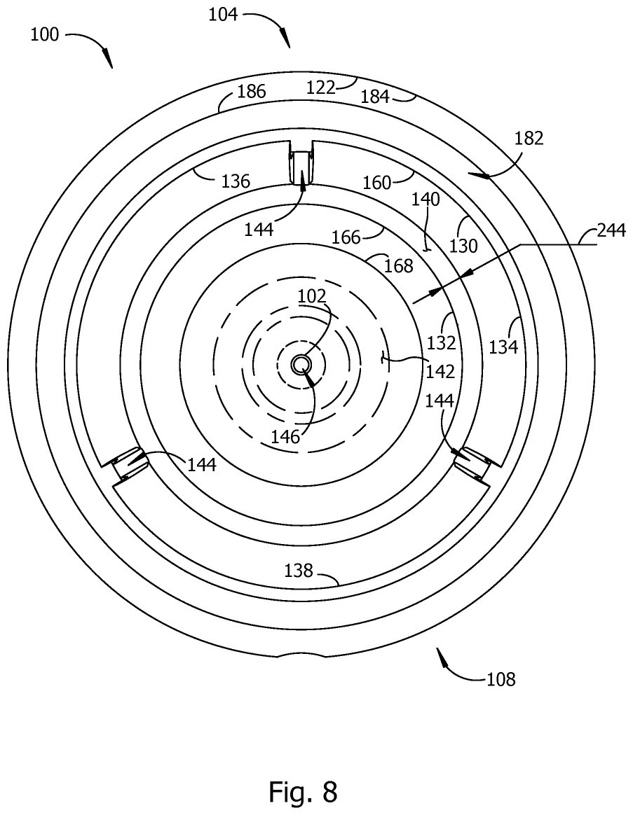

FIG. 8 shows the example microsurgical instrument 100 in a view toward the open proximal side 108 of the cannula head 104. The outer flange 122 of the cannula head is formed with an outer circumferential edge 184 and an inner circumferential edge 186. The inner flange 130 is spatially separated from the outer flange 122 by a gap 182 between the inner flange ridge 160 and the inner circumferential edge 186 of the outer flange 122. The illustrated example of the inner flange 130 includes a first inner flange segment 134, a second inner flange segment 136, and a third inner flange segment 138 separated from one another by inner flange slots 144. The payload guide 132 rests against the payload guide support wall 176 (not visible in this view), leaving a gap 244 between the outer circumferential edge 166 of the payload guide and the inner surface 140 of the inner flange 130. The inner surface 142 of the payload guide extends from an inner circumferential edge 168 at the proximal end of the payload guide to the spatulated cannula 102. The lumen 146 of the spatulated cannula 102 is visible at the center of the figure.

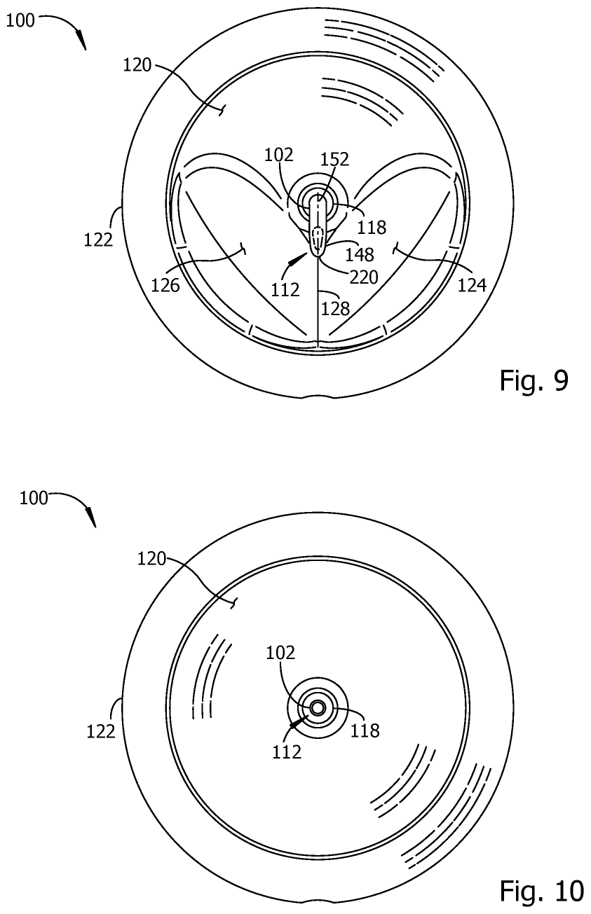

FIG. 9 shows an example of a cannula head 104 having a curved spatulated cannula 102 and an optional cannula orientation ridge 128 positioned to be coplanar with the cannula longitudinal centerline 152. FIG. 9 further illustrates an example of a curved spatulated cannula 102 with the arcuate segment 114 bent toward and in the same plane as the cannula orientation ridge, indicated in the figure by collinearity of the centerline 152 and the cannula orientation ridge. As shown in the figure, the example first inset face 124 and second inset face 126 intersect with one another along the cannula orientation ridge 128. An example of the parting tip projection extending out from the lumen of the cannula is also visible in FIG. 9. FIG. 10 illustrates an alternative embodiment 100 with a cannula head omitting the optional first inset face 124, second inset face 126, and cannula orientation ridge 128.

A side view of an example curved spatulated cannula 102 is shown in FIG. 11. As suggested in the illustrated example, the parting tip 112 is attached to the distal end of the arcuate segment 114 with a parting tip projection 148 along the top side 242 of the spatulated cannula. The arcuate segment 114 bends toward the bottom side 242. The distal end of the straight segment 116 attaches to the proximal end of the arcuate segment. For the illustrated example, the straight segment 116 has a length dimension 192 of about 21 millimeters, although other lengths may be selected. The bend radius 218 of the arcuate segment 114, extending from a center of curvature 230 to the exterior bottom side 242 of the spatulated cannula 102, is preferably within the range of values commonly found for the radius of Schlemm's canal as measured from the center of the pupil of the eye to an interior wall of Schlemm's canal, corresponding to a value in a range from 4.5 millimeters to 6.5 millimeters. The optional arcuate segment 114 of the spatulated cannula 102 subtends an arc angle 190 from the distal end of the straight segment to proximal end of the parting tip 112 at the proximal edge of the spatulated parting face 150, with the arc angle 190 preferably in a range from 20 degrees to 40 degree.

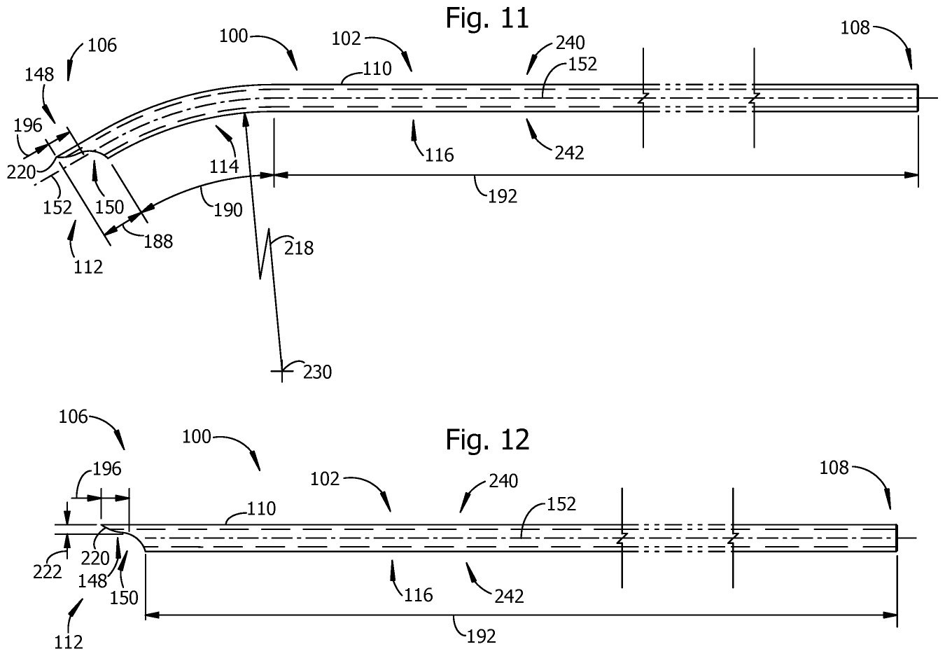

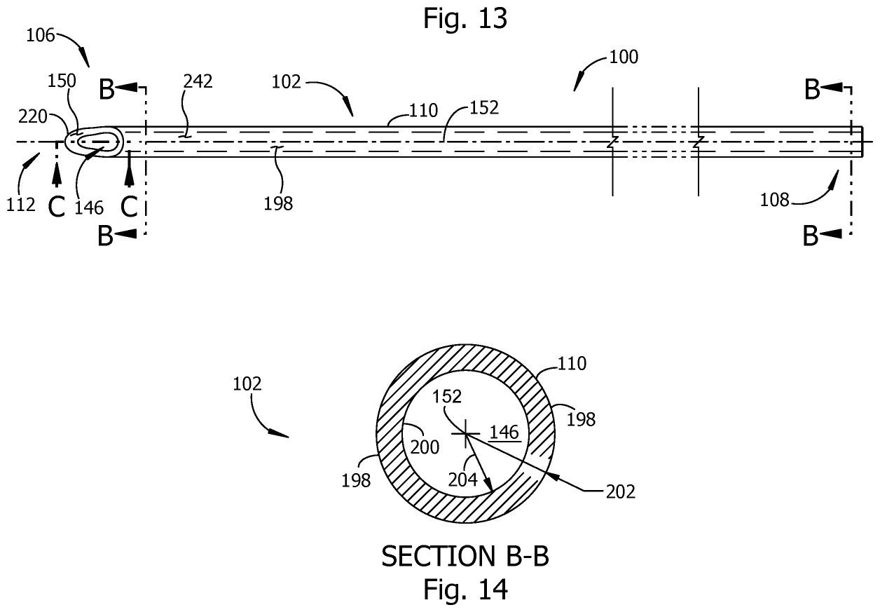

FIG. 12 shows a side view of an example straight spatulated cannula 102. In the example of FIG. 12, the proximal end of the parting tip, corresponding to the proximal end of the spatulated parting face 150, attaches directly to the straight segment 116 without the intervening arcuate segment of FIG. 11. FIG. 13 shows a spatulated cannula 102 in a view toward the parting face 150. FIG. 13 is representative of the view toward the bottom side of the curved spatulated cannula of FIG. 11 and the straight spatulated cannula of FIG. 12.

Both the straight and curved examples of the spatulated cannula 102 have a circular cross section everywhere from the proximal end 108 of the tubular member 110 to the proximal end of the parting tip 112. The inner radius 204 of the lumen 146, measured from the longitudinal centerline 152 of the spatulated cannula 102 to the interior surface 200 of the lumen as shown in FIG. 14, is preferably in a range from 0.125 millimeter to 0.175 millimeter, corresponding to a lumen diameter 208 in a range from 0.250 millimeter to 0.350 millimeter. The outer radius 202 of the tubular member 110, measured from the longitudinal centerline 152 to the exterior surface 198 of the spatulated cannula 102, is preferably in a range from 0.175 millimeters to 0.275 millimeters, with a maximum outer diameter of 0.55 millimeter.

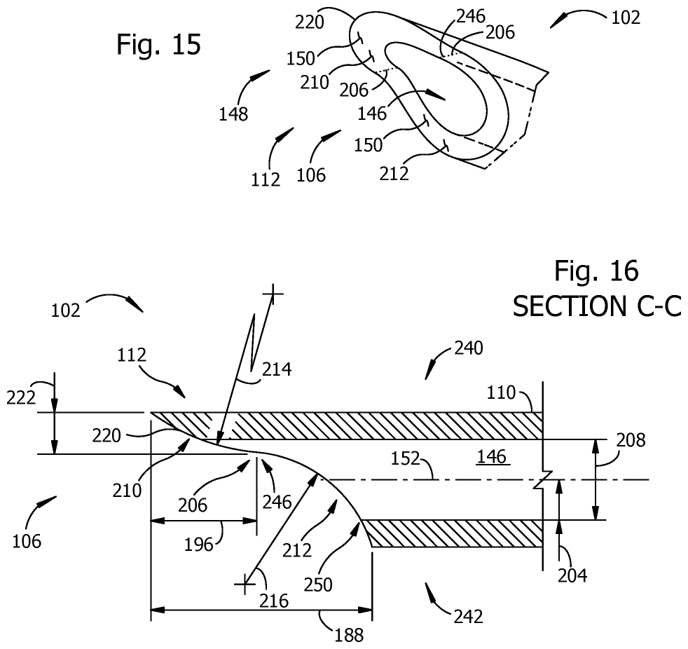

All example embodiments of the microsurgical instrument 100 include a spatulated cannula 102 with a preferred configuration of the spatulated parting face 150. As shown in the examples of FIG. 15 and FIG. 16, the preferred configuration of the spatulated parting face 150 of the parting tip 112 is a complex curved surface having a convex surface portion 210 joined to a concave surface portion 212 along a transverse line of inflection 206. The line of inflection establishes the proximal end of the parting tip projection and is the location of a transition in surface curvature from convex to concave on the spatulated parting face. The lumen 146 passes through the spatulated parting face 150 into the interior of the spatulated cannula 102. The intersection of the distal end of the convex surface portion 210 and the exterior surface 198 of the spatulated cannula 102 forms the convex parting edge 220 along transversely opposite sides of the parting tip projection 148. In the examples of FIGS. 15-16, the parting tip projection 148 and the convex parting edge 220 extend distally away from the line of inflection 206 to the extreme distal end of the spatulated cannula 102. The concave surface portion 212 extends proximally away from the line of inflection 206. The most proximal point of the intersection of the concave surface portion 212 with the exterior surface 198 on the bottom side 242 of the tubular member 110 establishes the proximal end of the parting tip 112.

The parting tip 112 of the preceding examples may be an integrally-formed element of the distal end of the spatulated cannula. The parting tip 112 may alternatively be formed as a separate hollow tubular element and joined by welding or adhesive to the tubular member 110 to form the spatulated cannula 102. The parting tip 112 may optionally be made from a different material than other parts of the spatulated cannula 102, for example a material better able to form the convex parting edge of the parting tip projection.

The following dimensions describe example implementations of a parting tip 112 for a spatulated cannula 102 suitable for introduction into Schlemm's canal. Dimensions other than those given in the examples may be used. Referring to the example of FIG. 16, the parting tip 112 has a length dimension 188 in a range from 0.7 millimeter to 1.5 millimeter, measured from the proximal end of the parting tip 112 to the distal end of the parting tip projection 148. In some embodiments, the length dimension 188 of the parting tip is 1.0 millimeter. A length dimension 196 of the parting tip projection, measured from the line of inflection 206 at the proximal end 246 of the parting tip projection to the distal extremity of the convex parting edge 220, has a minimum value of 0.2 millimeter. In some embodiments, the length dimension 196 of the parting tip projection is 0.4 millimeter. A thickness dimension 222 of the parting tip projection 148, measured from the line of inflection 206 to the convex parting edge 220 at the top 240 of the exterior surface 198 of the tubular member 110, has a value in a range from 0.125 millimeter to 0.275 millimeter. In some embodiments, the thickness dimension 222 of the parting tip projection is 0.15 millimeter. A spatulation radius 216 for the concave portion 212 of the spatulated parting face 150 has a value in a range from 0.3 millimeter to 1.0 millimeter. A radius 214 for the convex portion 210 of the spatulated parting face 150 has a value greater than or equal to 0.4 millimeter. In some embodiments, the radius 214 for the convex portion 210 is 0.8 millimeter.

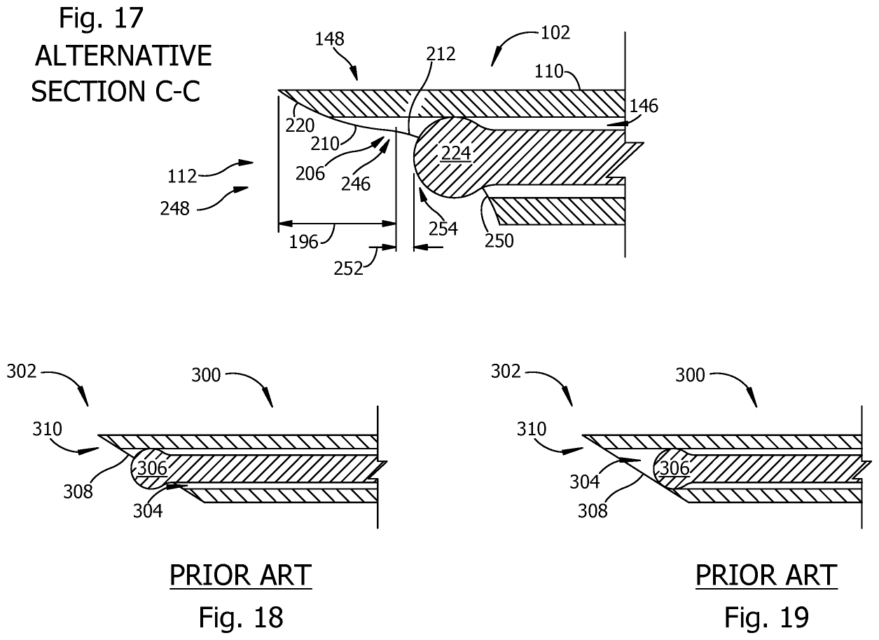

A microsurgical instrument 100 having a spatulated parting face 150 provides enhanced visibility of the distal end of the spatulated cannula compared to previously known cannulas. As suggested in the examples of FIG. 16 and the alternative cross-section C-C in FIG. 17, the length dimension 196 of the parting tip projection 148 relative to the spatulation radius 216 and the diameter 208 of the lumen 146 enable a distal end 254 of a light source 224 to protrude outward from the distal edge 250 of the lumen in an example enhanced visibility position 248, without the distal end of the light source extending over or beyond the proximal end 246 of the parting tip projection 148. By at least partially extending outside the lumen, light emitted from the distal end of the light source 224 is not blocked by the walls of the lumen in many directions and is reflected from the polished concave surface portion 212 of the parting tip 112. The enhanced visibility position 248 preferably includes a separation distance 252 between the proximal end 246 of the parting tip projection 148 and the distal end 254 of the light source 224. Furthermore, by not extending over or past the parting tip projection, that is, the separation distance 252 has a value greater than or equal to zero, the light source does not interfere with the effectiveness of the parting tip projection to penetrate tissue such as the sclera of an eye. The entire length 196 of the parting tip projection remains available for tissue penetration, and the position of the parting tip through obscuring tissue such as the sclera of an eye is made dearly visible over a wide range of viewing directions by the bright light emission from the enhanced visibility position of the light source.

As suggested in the prior art example of FIG. 18, a light source 306 may be extended from the lumen 304 through a bevel face 310 at the distal end 302 of a cannula 300 previously known in the art. However, in contrast to the disclosed microsurgical instruments 100, the farther the light source extends from the bevel face 310, the more the distal end of the light source 306 interferes with the effectiveness of the cutting edge 308 for penetrating tissue. For some extension distances, the protruding light source limits penetration of the cutting edge to a small fraction of the cannula diameter, possibly preventing passage of the cannula 300 through a tissue surface or possibly causing tissue damage as the cannula is forced through an insufficiently large incision made by the distal tip of the cutting edge 308. To avoid tissue damage and/or bending of the cannula 300, the light source 306 may then be retracted into the lumen 304 as in the example of FIG. 19, restoring the ability of the cutting edge to penetrate tissue but reducing the range of directions over which light from the light source is visible.

Unless expressly stated otherwise herein, ordinary terms have their corresponding ordinary meanings within the respective contexts of their presentations, and ordinary terms of art have their corresponding regular meanings.

What is claimed is:

1. A cannula formed as a hollow tube, comprising:
a straight segment at a proximal end of said cannula; and
a parting tip at a distal end of said cannula, said parting tip comprising:
a parting tip projection;
a convex parting edge formed on said parting tip projection; and
a spatulated parting face, comprising:
a convex surface portion formed on said parting tip projection; and
a concave surface portion joined to said convex surface portion along a line of inflection at a proximal end of said parting tip projection.

2. The cannula of claim 1, further comprising said convex surface portion of said spatulated parting face extending from said convex parting edge to said line of inflection.

3. The cannula of claim 1, further comprising said concave surface portion extending to an external surface of said cannula.

4. The cannula of claim 1, wherein said concave surface portion is formed with a spatulation radius having a value in a range from 0.3 millimeter to 1.0 millimeter.

5. The cannula of claim 1, wherein said convex surface portion is formed with a radius greater than or equal to 0.4 millimeter.

6. The cannula of claim 1, wherein said cannula is formed with a lumen through said straight segment and said parting tip, said lumen having a diameter with a value in a range from 0.250 millimeter to 0.350 millimeter.

7. The cannula of claim 1, wherein a length dimension of said parting tip projection from said line of inflection to said convex parting edge has a minimum value of 0.2 millimeter.

8. The cannula of claim 1, wherein a thickness dimension of said parting tip projection has a value in a range from 0.125 millimeter to 0.275 millimeter.

9. The cannula of claim 1, wherein said straight segment and said parting tip are integrally formed with one another.

10. The cannula of claim 1, further comprising an arcuate segment interposed between said parting tip and said straight segment, said arcuate segment extending away from said straight segment over an arc angle having a value in a range from 20 degrees to 40 degrees.

11. The cannula of claim 1, wherein said arcuate segment is formed with a bend radius having a value in a range from 4.5 millimeters to 6.5 millimeters.

12. A microsurgical instrument, comprising:
a spatulated cannula formed as a hollow tube, comprising:
a straight segment at a proximal end of said cannula; and
a parting tip at a distal end of said cannula, said parting tip having a spatulated parting face;
a cannula head, comprising:
an exterior wall having a tapered outer surface;
a circumferential outer flange attached to said outer surface at a proximal end of said cannula head;
an arcuate inner flange extending proximally away from said exterior wall, said inner flange comprising:
an outer surface; and
an inner flange ridge extending radially away from said outer surface;
a payload guide support wall joined to said exterior wall; and
a payload guide attached to said spatulated cannula, said payload guide slidably engaged with said payload guide support wall.

13. The microsurgical instrument of claim 12, further comprising a circumferential channel formed between said outer flange and said inner flange ridge.

14. The microsurgical instrument of claim 12, wherein said payload guide support wall and said payload guide are each formed with a Morse taper.

15. The microsurgical instrument of claim 12, said parting tip further comprising:
a parting tip projection; and
a convex parting edge formed on said parting tip projection.

16. The microsurgical instrument of claim 12, said parting tip further comprising:
a parting tip projection; and
a spatulated parting face, comprising:
a convex surface portion formed on said parting tip projection; and
a concave surface portion joined to said convex surface portion along a line of inflection at a proximal end of said parting tip projection.

17. The microsurgical instrument of claim 12, said cannula head further comprising:
a first inset face formed on said exterior wall;
a second inset face formed on said exterior wall; and
a cannula orientation ridge positioned along an intersection of said first inset face and said second inset face.

18. The microsurgical instrument of claim 17, further comprising an arcuate segment interposed between said parting tip and said straight segment of said spatulated cannula.

19. The microsurgical instrument of claim 18, wherein a longitudinal centerline through said arcuate segment is coplanar with said cannula orientation ridge.

20. The microsurgical instrument of claim 12, further comprising:
a light source; and
an enhanced visibility position of said spatulated cannula, comprising:
a distal end of said light source extending outward from a distal edge of said lumen; and
a separation distance between a proximal end of said parting tip projection and said distal end of said light source.
